(19) United States
(12) Patent Application Publication
(10) Pub. No.: US 2018/0263945 A1
(43) Pub. Date: Sep. 20, 2018

(54) PYRUVATE COMPOUNDS FOR TREATMENT OF PERIPHERAL NEUROPATHY

(71) Applicant: Research Institute at Nationwide Children's Hospital, Columbus, OH (US)

(72) Inventor: ZARIFE SAHENK, Columbus, OH (US)

(21) Appl. No.: 15/537,589

(22) PCT Filed: Dec. 21, 2015
(86) PCT No.: PCT/US15/66999

§ 371 Date: Jun. 19, 2017

Related U.S. Application Data
(60) Provisional application No. 62/094,323, filed on Dec. 19, 2014.

(51) Int. Cl. A61K 31/22; A61K 48/00; A61K 9/00; A61P 25/02

(52) U.S. Cl. CPC A61K 31/22; A61P 25/02; A61K 9/0053; A61K 48/0083

(57) ABSTRACT

A method of treating a subject having peripheral neuropathy is described that includes administering a therapeutically effective amount of a pyruvate compound to the subject. Controlled release formulations of a pharmaceutically acceptable pyruvate salt can be used to administer the pyruvate compound over a substantial period of time.

### CROSS-REFERENCE TO RELATED APPLICATIONS

This application claims priority to U.S. Provisional Application Ser. No. 62/094,323, filed on Dec. 19, 2014, which is hereby incorporated by reference in its entirety.

### BACKGROUND

Neuropathological studies of experimental toxic neuropathies from 1970s unveiled an important feature of axonal degeneration. During the evolution of “dying back” axonopathy, distal segments of long axons respond in a stereotypical manner under many diverse and seemingly unrelated detrimental conditions. The disruption of energy-dependent axonal transport system as the mechanisms underlying this length-dependent distal axonal degeneration, first proposed more than thirty years ago by Spencer et al (Spencer et al., Ann Neurol 5, 501-507 (1979)) is now revisited with renewed enthusiasm and a slightly different perspective, from the angle of glia-axon interactions. Brown et al., Ann Neurol 72, 406-418 (2012).

Most Charcot-Marie-Tooth (CMT) patients including those with primary Schwann cell genetic defects present with a clinical phenotype of length-dependent axonal disease. Previous studies have shown that axonal pathology in “demyclinating” CMT neuropathies is an important feature that directly correlates with the clinical disability. Sahenk, Z., and Chen, L., J Neurosci Res 51, 174-184 (1998); Sahenk et al., Ann Neurol 45, 16-24 (1999). Profound axonal cytoskeletal abnormalities leading to axonal degeneration and preferential axonal loss seen in trembler^{J} (Tr^{J}) mice and in xenografts from patients with primary Schwann cell genetic defects are thought to result from impaired Schwann cell-axon interactions. de Waegh et al., Cell 68, 451-463 (1992); Sahenk, Z., Ann N Y Acad Sci 883, 415-426 (1999). improving the hypomyelination/amyelination state, which is the hallmark of trembler pathology.

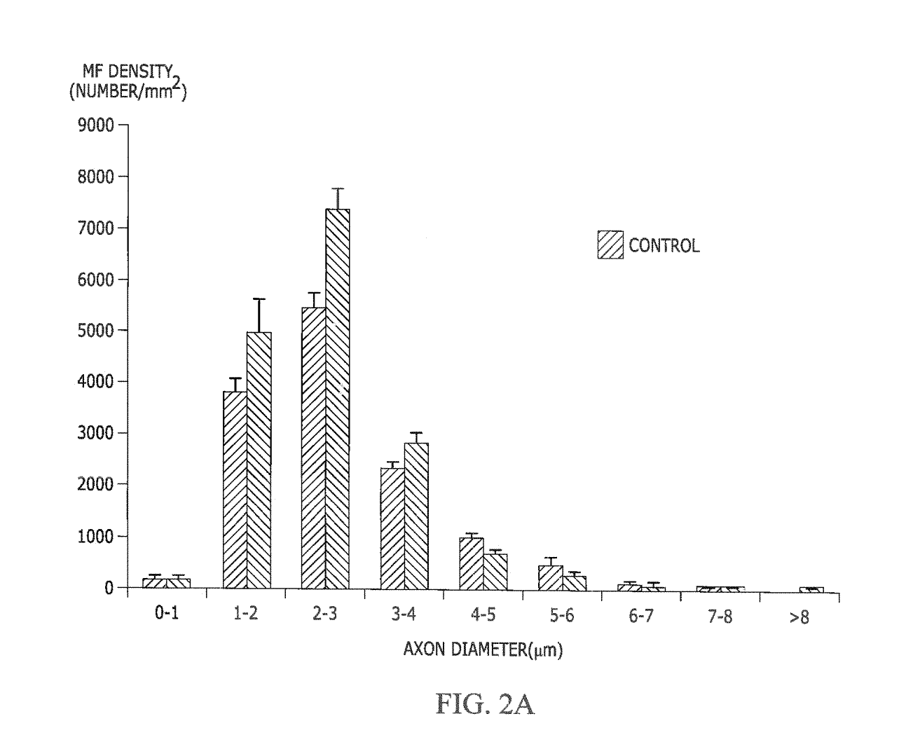

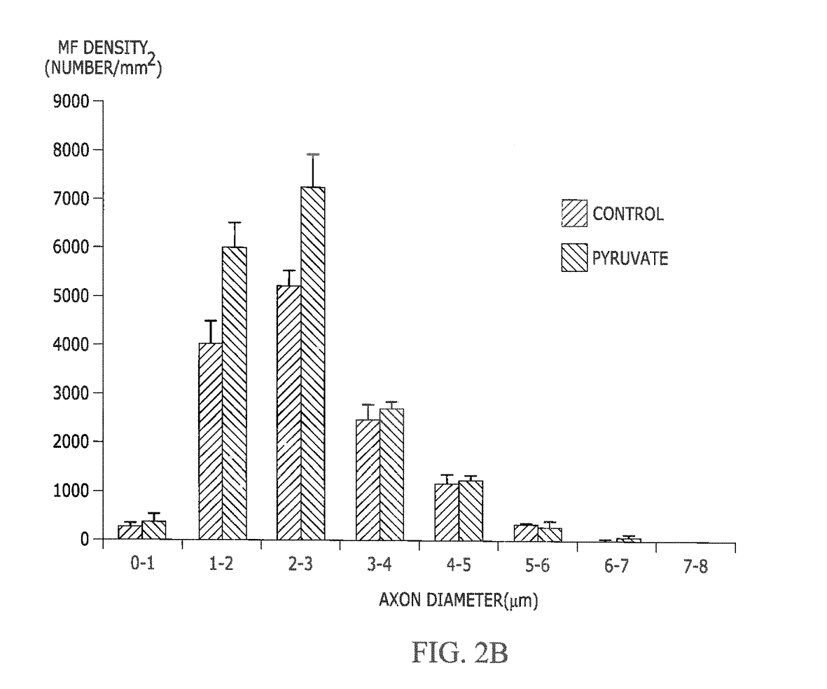

FIGS. 2A and 2B provide composite histograms generated from pyruvate (n=5) and control (n=3) Tr^{J} mice. Both in the intact (A) and regenerating (B) nerves, the increase in myelinated fiber densities are most prominent for those fibers with axonal diameter less than 4 μm. (In the pyruvate group, a total of 2583 measurements in the intact/uncrushed nerves and 2844 in the regenerating nerves were made, derived from 5 mice. In the control group, 1271 measurements in the intact and 1106 in the regenerating nerves were obtained, derived from 3 mice. The myclinated fiber (MF) densities were expressed as number per mm² of the endoneurial area).

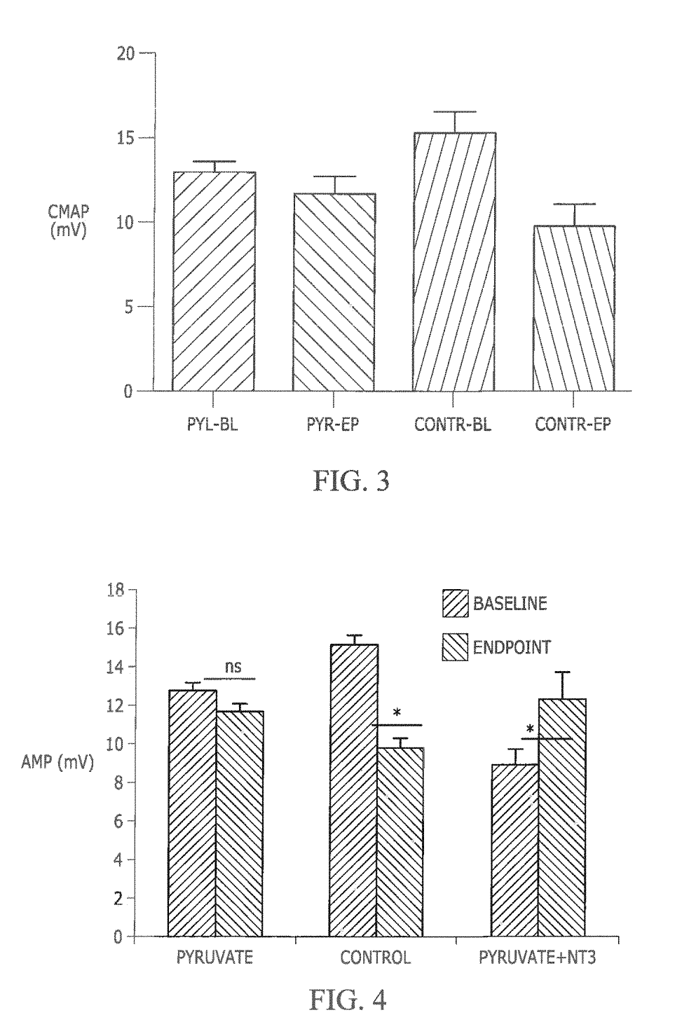

FIG. 3 provides bar graphs showing that exogenous pyruvate protects compound muscle action potentials from further decline in trembler mice.

FIG. 4 provides a bar graph showing the results of nerve conduction studies that were performed at baseline and endpoint following 4 months of treatment duration. CMAPS were shown to decrease in control group (n=6, P<0.05) while pyruvate treated mice preserved their CMAPs during the treatment period (n=6). Pyruvate+NT-3 group had significantly improved CMAP amplitudes at the endpoint compared to the baseline (n=10, P<0.05). (Error bars represent standard error of the mean. Student t test was performed).

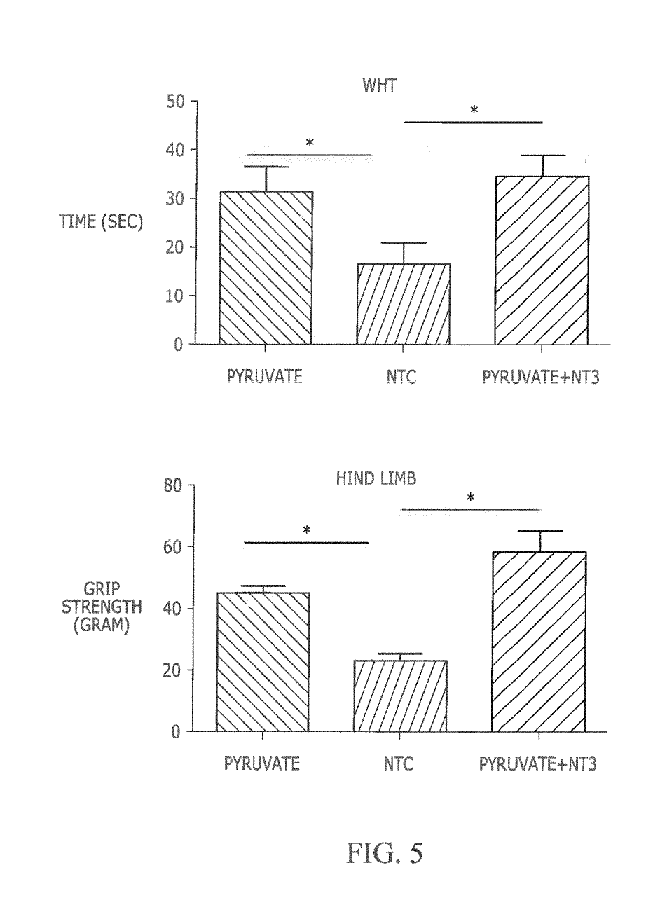

FIG. 5 provides bar graphs comparing the results of combination therapy with those obtained using pyruvate alone. Mice were tested for motor functions at the endpoint (4 months post injection) by performing grip strength test and four-limb wire hanging strength test. Both pyruvate (n=6) and pyruvate+NT-3 (n=10) treated group performed better than control group (n=6) in both test (*P<0.05). (Error bars represent standard error of the mean. Student t test was performed).

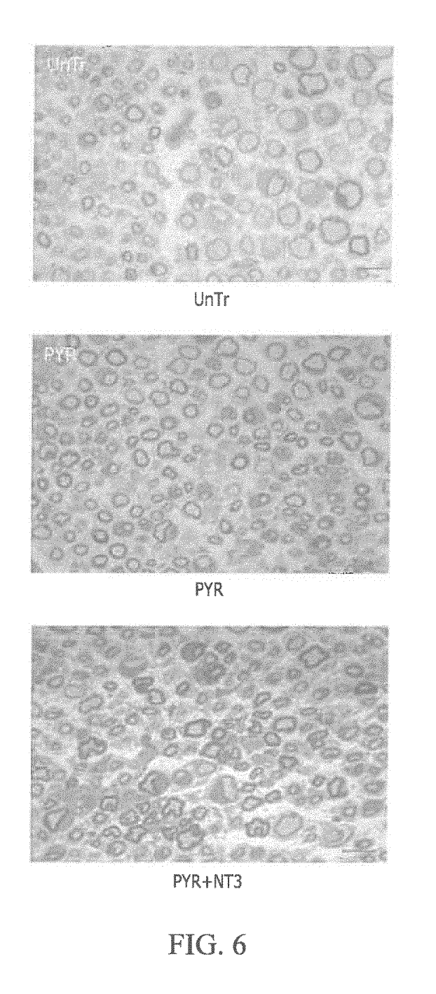

FIG. 6 provides images of μm thick toluidine blue stained plastic sections from sciatic nerves of TrJ, untreated (UnTr), treated with SP alone (PYR) and received the combination of AAV1.NT-3 gene therapy and SP (PYR+NT3) at 16 weeks of treatment. Untreated TrJ mice nerves show severe hypomyelination. A notable increase in myelinated fibers and increased myelin thickness is seen in both treatment groups, more prominent in the nerves received the combination therapy.

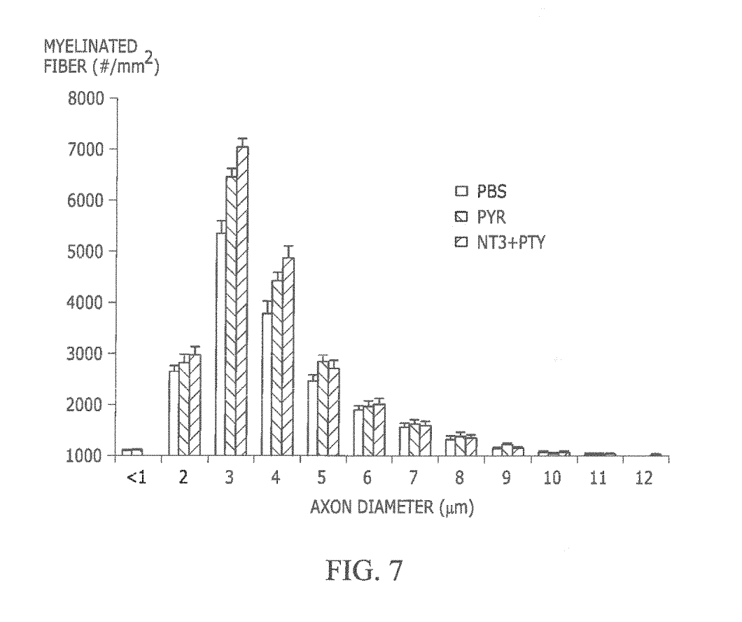

FIG. 7 provides a composite histogram showing myelinated fiber distribution in the treated and untreated sciatic nerves from TrJ mice at 16 weeks of treatment. Quantitative analysis at the light microscopic level was performed on 1 pun-thick cross sections from sciatic nerves, photographed at ×100 using an image analysis soft ware (Bioquant TCW14 image analysis software, R&M Biometrics Inc., Nashville, Tenn.) as previously described. Sahenk et al., J Peripher Nerv Syst 8: 116-127 (2003). Four random areas were photographed in each nerve. Composites of fiber size distribution histograms and mean myelinated fiber (MF) densities (number per mm² of fascicular area) were generated by combining data from six mice in each cohort.

### DETAILED DESCRIPTION OF THE INVENTION

The present invention relates to methods of treating a subject having peripheral neuropathy (e.g., Charcot-Marie-Tooth neuropathy) by administering a therapeutically effective amount of a pyruvate compound to the subject.

### Definitions

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

Prevention, as used herein, refers to any action providing a benefit to a subject at risk of being afflicted with a condition or disease such as Charcot-Marie-Tooth neuropathy, including avoidance of infection or a decrease of one or more symptoms of the disease should infection occur.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of an agent which will achieve the goal of improvement in disease severity and the frequency of incidence. The effectiveness of treatment may be measured by evaluating a reduction in psychotic symptoms in a subject in response to the administration of antipsychotic agents.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

The term “gene” as used herein refers to a nucleotide sequence that can direct synthesis of an enzyme or other polypeptide molecule (e.g., can comprise coding sequences, for example, a contiguous open reading frame (ORF) which encodes a polypeptide) or can itself be functional in the organism. A gene in an organism can be clustered within an operon, as defined herein, wherein the operon is separated from other genes and/or operons by intergenic DNA. Individual genes contained within an operon can overlap without intergenic DNA between the individual genes.

The term “vector” or “expression vector” refers to any type of genetic construct comprising a nucleic acid coding for an RNA capable of being transcribed. Expression vectors can contain a variety of control sequences, structural genes (e.g., genes of interest), and nucleic acid sequences that serve other functions as well.

As used herein, the term “about” refers to +/−10% deviation from the basic value.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a pyruvate compound” also includes a plurality of such compounds.

### Use of Pyruvate Compounds

One aspect of the invention provides a method of treating a subject having peripheral neuropathy. The method comprises administering a therapeutically effective amount of a pyruvate compound to the subject. Pyruvate compounds, as used herein, include both the conjugate base pyruvate (CH₃COCOO—) and pyruvic acid (CH₃COCOOH). Pyruvate is the simplest of the alpha-keto acids, with a carboxylic acid and a ketone functional group, and is a key intermediate in several metabolic pathways.

In some embodiments, the pyruvate compound is a pharmaceutically acceptable salt of pyruvate. Pharmaceutically acceptable salt refers to the relatively non-toxic, inorganic and organic acid addition salts of the compounds. These salts can be prepared in situ during the final isolation and purification of the compound, or by separately reacting pyruvate with a suitable counterion and isolating the salt thus formed. Representative countrions include sodium, potassium, calcium, magnesium, ammonium, arginine, diethylamine, ethylenediamine, and piperazine salts, and the like. See for example Haynes et al., J. Pharm. Sci., 94, p. 2111-2120 (2005). For example, in some embodiments, the pyruvate compound is selected from the group consisting of sodium pyruvate, calcium pyruvate, potassium pyruvate, and magnesium pyruvate. A preferred pyruvate compound is sodium pyruvate.

In some embodiments, the pyruvate compound is a pyruvate alkyl ester derivative. Pyruvate alkyl ester derivatives are forms of pyruvic acid in which an alkyl group is attached to the non-carbonyl oxygen of the carboxylic acid group. “Pyruvic acid alkyl ester,” “alkyl ester of pyruvic acid” and like terms refer to compounds of Formula I, and all tautomcric and charged forms thereof,

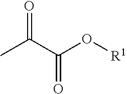

wherein R¹ is alkyl.

The term “alkyl” refers to straight, branched chain, or cyclic hydrocarbyl groups including from 1 to about 20 carbon atoms. Alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups. Preferred alkyl groups include alkyl groups having from 1 to 6 carbon atoms, more preferred alkyl groups have 2 carbon atoms (i.e., ethylpyruvate).

### Treatment of Peripheral Neuropathy

The present invention provides a method of treating a subject having peripheral neuropathy. Peripheral neuropathy is damage to or disease affecting peripheral nerves, which may impair sensation, movement, gland or organ function, or other aspects of health, depending on the type of nerve affected. Common causes include systemic diseases (such as diabetes or leprosy), vitamin deficiency, medication (e.g., chemotherapy), traumatic injury, radiation therapy, excessive alcohol consumption, immune system disease or viral infection. It can also be genetic (present from birth) or idiopathic. Peripheral neuropathy may be classified according to the number and distribution of nerves affected (mononeuropathy, mononeuritis multiplex, or polyneuropathy), the type of nerve fiber predominantly affected (motor, sensory, autonomic), or the process affecting the nerves; e.g., inflammation (neuritis), compression (compression neuropathy), chemotherapy (chemotherapy-induced peripheral neuropathy).

In some embodiments, the subject has been diagnosed as having a disorder of the peripheral nervous system (e.g., peripheral neuropathy). Symptoms of peripheral nervous system disorders include, pain and parasthesia that appears symmetrically and generally at the terminals of the longest nerves, which are in the lower legs and feet. Sensory symptoms generally develop before motor symptoms such as weakness. Length-dependent peripheral neuropathy symptoms make a slow ascent of leg, while symptoms may never appear in the upper limbs. Peripheral neuropathy may first be considered when an individual reports symptoms of numbness, tingling, and pain in feet. In further embodiments, the subject has been diagnosed as exhibiting muscle weakness, atrophy and/or sensory dysfunction.

In some embodiments, the peripheral neuropathy is Charcot-Marie-Tooth neuropathy. Charcot-Marie-Tooth neuropathy (CMT), also known as Charcot-Mari-Tooth disease, hereditary motor and sensory neuropathy (HMSN) and peroneal muscular atrophy (PMA), is a genetically and clinically heterogeneous group of inherited disorders of the peripheral nervous system characterized by progressive loss of muscle tissue and touch sensation across various parts of the body. Loss of touch sensation in the feet, ankles and legs, as well as in the hands, wrists and arms occur with various types of the disease. Early and late onset forms occur with ‘on and off’ painful spasmodic muscular contractions that can be disabling when the disease activates. High arched feet or flat arched feet are classically associated with the disorder. CMT can be diagnosed through symptoms, through measurement of the speed of nerve impulses (nerve conduction studies), through biopsy of the nerve, and through DNA testing. DNA testing is preferred, and can give a definitive diagnosis.

Pyruvate compounds can be used to provide prophylactic and/or therapeutic treatment. Pyruvate compounds can, for example, be administered prophylactically to a subject in advance of the occurrence of peripheral neuropathy. Prophylactic (i.e., preventive) administration is effective to decrease the likelihood of the subsequent occurrence of peripheral neuropathy in the subject, or decrease the severity of peripheral neuropathy that subsequently occurs. Prophylactic treatment may be provided to a subject that is at elevated risk of developing peripheral neuropathy, such as a subject with a family history of peripheral neuropathy. The expression of mutations of myelin protein 22 (PMP22) represents 70-80% of all occurrences of Charcot-Marie-Tooth neuropathy, and thus their presence may be useful as criteria for selecting patients to receive treatment using the pyruvate compounds described herein.

Alternatively, the compounds of the invention can be administered therapeutically to a subject that is already afflicted by peripheral neuropathy. In one embodiment of therapeutic administration, administration of the compounds is effective to eliminate the peripheral neuropathy, in another embodiment, administration of the pyruvate compounds is effective to decrease the severity of the peripheral neuropathy or lengthen the lifespan of the subject so afflicted. In some embodiments, the method of treatment consists of administering a therapeutically effective amount of a pyruvate compound in a pharmaceutically acceptable formulation to the subject over a substantial period of time.

### Gene Therapy of Peripheral Neuropathy

In one aspect, the present invention provides methods of treating a subject having peripheral neuropathy that includes the combined use of gene therapy and administering a therapeutically effective amount of a pyruvate compound to the subject over a substantial period of time. Preferably, the gene therapy is carried out first, followed by administration of a pyruvate compound over a substantial period of time.

Vectors which can be used to deliver a therapeutic nucleic acid include viral and non-viral vectors. Suitable vectors which can be used include adenovirus, adeno-associated virus, retrovirus, lentivirus, HSV (herpes simplex virus) and plasmids. An advantage of Herpes simplex virus vectors is their natural tropism for sensory neurons. However, adenovirus associated viral vectors are most popular, due to their low risk of insertional mutagenesis and immunogenicity, their lack of endogenous viral genes, and their ability to be produced at high titer. Kantor et al. review a variety of methods of gene transfer to the central nervous system, while Goins et al. describe methods of gene therapy for the treatment of chronic peripheral nervous system pain. See Kantor et al., Adv Genet. 87, 125-197 (2014), and Goins et al., Neurobiol. Dis. 48(2), 255-270 (2012), the disclosures of which are incorporated herein by reference. In particular, successful gene delivery to Schwann cells, the resident glia cells of pierphal nerves, has been reported using various viral vectors. Mason et al., Curr. Gene Ther. 11, 75-89 (2011). If the vector is in a viral vector and the vector has been packaged, then the virions can be used to infect cells. If naked DNA is used, then transfection or transformation procedures as are appropriate for the particular host cells can be used. Formulations of naked DNA utilizing polymers, liposomes, or nanospheres can be used for gene delivery. Nucleic acids can be administered in any desired format that provides sufficiently efficient delivery levels, including in virus particles, in liposomes, in nanoparticles, and complexed to polymers.

The nucleic acid (e.g., cDNA or transgene) encoding a gene whose expression decreases peripheral neuropathy can be cloned into an expression cassette that has a regulatory element such as a promoter (constitutive or regulatable) to drive transgene expression and a polyadenylation sequence downstream of the nucleic acid. For example, regulatory elements that are 1) specific to a tissue or region of the body; 2) constitutive; and/or 3) inducible/regulatable can be used.

In some embodiments, muscle-specific regulatory elements are used. Muscle-specific regulatory elements include muscle-specific promoters including mammalian muscle creatine kinase (MCK) promoter, mammalian desmin promoter, mammalian troponin I (TNNI2) promoter, or mammalian skeletal alpha-actin (ASKA) promoter. Muscle-specific enhancers useful in the present invention are selected from the group consisting of mammalian MCK enhancer, mammalian DES enhancer, and vertebrate troponin I IRE (TNI IRE, herein after referred to as FIRE) enhancer. One or more of these muscle-specific enhancer elements may be used in combination with a muscle-specific promoter of the invention to provide a tissue-specific regulatory element.

A preferred vector for use in treating peripheral neuropathy by gene therapy is AAV. AAV-mediated gene delivery has emerged as an effective and safe tool for both preclinical and clinical studies of neurological disorders. Ojala et al., Neuroscientist., 21(1):84-98 (2015). Currently, AAV is the most widely used vector for clinical trials for neurological disorders, and no adverse effects linked to the use of this vector have ever been reported from clinical trials. Adeno-associated virus is a non-pathogenic dependovirus from the parvoviridae family requiring helper functions from other viruses, such as adenovirus or herpes simplex virus, to fulfill its life cycle. The wild-type (WT) AAV is characterized by a single-stranded DNA (ssDNA) genome, with inverted terminal repeats (ITR) at both ends, of approximately 5 kb surrounded by a capsid.

Adenoviral vectors for use to deliver transgenes to cells for applications such as in vivo gene therapy and in vitro study and/or production of the products of transgenes, commonly are derived from adenoviruses by deletion of the early region 1 (E1) genes (Berkner, K. L., Curr. Top. Micro. Immunol. 158 L39-66 1992). Deletion of E1 genes renders such adenoviral vectors replication defective and significantly reduces expression of the remaining viral genes present within the vector. Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts. However, it is believed that the presence of the remaining viral genes in adenoviral vectors can be deleterious.

Accordingly, in some embodiments, adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (See, U.S. Pat. No. 5,882,877 which covers pseudoadenoviral vectors (PAV) and methods for producing PAV, incorporated herein by reference). Such PAVs, which can accommodate up to about 36 kb of foreign nucleic acid, are advantageous because the carrying capacity of the vector is optimized, while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis-acting nucleotide sequence required for packaging of the PAV genome, and can accommodate one or more transgenes with appropriate regulatory elements, e.g. promoter, enhancers, etc.

AAV of any serotype can be used. The serotype of the viral vector used in certain embodiments of the invention is selected from the group consisting from AAV1, AAV2, AAV3, AAV4, AAV5, AAV6, AAV7, AAV8, AAV9, AAV10, AAV11, AAV12 or AAV13 (see, e.g., Gao et al., PNAS, 99:11854-11859 ((2002); and Viral Vectors for Gene Therapy: Methods and Protocols, ed. Machida, Humana Press, 2003). Other serotype besides those listed herein can be used. For example, AAV vectors having novel serotypes can be designed using a combinatorial capsid library to provide vectors having substantially increased transduction efficiency. Marsic et al., Mol Ther. 22(11):1900-9 (2014). Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome. (Auricchio et al., (2001) Hum. Mol. Genet., 10 (26):3075-81.)

Gene therapy can be used together with administration of a pyruvate compound to treat peripheral neuropathy. The gene targeted by the gene therapy should be a gene whose expression decreases peripheral neuropathy. A wide variety of genes are known by those skilled in the art to be involved in various different types of peripheral neuropathy. For example, Jun Li describes a variety of genes involved in various forms of inherited neuropathies. See Jun Li, Semin Neurol. 32, 204-214 (2012). A number of different genes have been identified as being involved in various different forms of CMT which can be used in gene therapy treatment. Mathis et al., Expert Rev. Neurother. 15, 355-66 (2015). In some embodiments, the gene therapy treatment increases neurotrophin-3 (NT-3) expression, since NT-3 plays a significant role in CMT1, which is a particular variant of CMT. The nucleotide sequence for the NT-3 gene is known. See Accession No. AC007848.

In one embodiment, the gene therapy is NT-3 gene therapy via recombinant adeno-associated virus (AAV) delivery. The inventors developed an AAV expression cassette carrying human NT-3 cDNA coding sequence under the control of either the CMV promoter or triple muscle-specific creatine kinase (tMCK) promoter. The inventors have previously shown that an improvement in motor function, histopathology, and electrophysiology of peripheral nerves can be achieved using the recombinant AAV1 vector to increase neurotrophin-3 expression in the trembler^{J} (Tr^{J}) mouse, which is a model for the Charcot-Marie-Tooth disease variant CMT1A. See Sahenk et al., Mol Ther. 22(3):511-21 (2014), the disclosure of which is incorporated herein by reference.

### Administration and Formulation

The present invention also provides pharmaceutical compositions that include pyruvate compounds as an active ingredient, and a pharmaceutically acceptable liquid or solid carrier or carriers, in combination with the active ingredient. Any of the pyruvate compounds described herein as suitable for the treatment of peripheral neuropathy can be included in pharmaceutical compositions of the invention.

The pharmaceutical compositions include one or more pyruvate compounds together with one or more of a variety of physiological acceptable carriers for delivery to a subject, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the pyruvate compound, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the pyruvate compound into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The formulations include, but are not limited to, those suitable for oral, inhaled, rectal, vaginal, topical, nasal, ophthalmic, or parenteral (including subcutaneous, intramuscular, intraperitoneal, and intravenous) administration.

Pharmaceutical compositions and preparations typically contain at least about 0.1 wt-% of the active agent. The amount of the pyruvate compound is such that the dosage level will be effective to produce the desired result in the subject. Useful dosages of the pyruvate can be determined by comparing their in vitro activity and their in vivo activity in animal models. Methods for extrapolation of effective dosages in mice, and other animals, to humans are known in the art; for example, see U.S. Pat. No. 4,938,949.

Formulations of the present invention suitable for oral administration may be presented as discrete units, each containing a predetermined amount of the active agent as a powder or granules, as liposomes containing the active compound, or as a solution or suspension in an aqueous liquor or non-aqueous liquid such as a syrup, an elixir, an emulsion, or a draught. Inhaled formulations include those designed for administration from an inhaler device. Compositions for inhalation or insufflation include solutions and suspensions in pharmaceutically acceptable, aqueous or organic solvents, or mixtures thereof, aerosols, and powders. Preferably, the compositions are administered by the oral or nasal respiratory route for local or systemic effect. Compositions in preferably pharmaceutically acceptable solvents may be nebulized by use of inert gases. Solution, suspension, or powder compositions may be administered, preferably orally or nasally, from devices that deliver the formulation in an appropriate manner. Nasal spray formulations include purified aqueous solutions of the active agent with preservative agents and isotonic agents. Such formulations are preferably adjusted to a pH and isotonic state compatible with the nasal mucous membranes.

Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

The pyruvate compounds can be administered as a single dose or in multiple doses. In some embodiments, the pyruvate compound is administered in a plurality of separate administrations. For example, the pyruvate compound can be administered twice, three times, four times, five times, about ten times, about twenty times, or more than 20 times to the subject. In further embodiments, the pyruvate is administered to the subject on an hourly, daily, or weekly basis. Because pyruvate compounds typically do not persist for a lengthy period within a subject, in some embodiments, the pyruvate compound is administered over a substantial period of time to continue to provide treatment for peripheral neuropathy. For example, the pyruvate compound can be administered for about a week, the compound can be administered for about a month, or the compound can be administered for a year or more.

### Controlled Release Formulations

In some embodiments, the pyruvate compound is administered in a controlled release formulation. A wide variety of controlled release formulations are known to those skilled in the art. Many controlled release formulations are based on the use of biodegradable and/or biocompatible pharmaceutical polymers. These include polyester-based synthetic polymers, and natural-origin polymers. Examples of polyester-based synthetic polymers include PLGA, poloxamer, polyvinylpyrrolidone ethylcellulose, sodium pyrrolidone carboxylate, povidone, polylactic acid (PLA), poly(ethylene glycol) (PEG), polyvinyl alcohol (PVA), and mixtures thereof. Examples of natural-origin polymers include starch, hyaluronate, human albumin, gelatin, alginic acid, and collagen. Alternately, or in addition to the use of pharmaceutical polymers, controlled release delivery can be achieved through the use of microparticles or nanoparticles. See Mansour et al., Int. J. Mol. Sci. 11, 3298-3322 (2010) for further information on materials for materials suitable for use in controlled release drug delivery formulations.

Use of a controlled release formulation of the pyruvate compound facilitates administration of the compound over a substantial period of time. In some embodiments, the controlled release formulation includes a pharmaceutically acceptable pyruvate salt. In another embodiment, the pyruvate compound is administered as a pharmaceutically acceptable salt that is orally administered in a controlled release formulation.

### Food Additives and Dietary Supplements

In some embodiments, the present invention provides compositions for oral ingestion comprising a pyruvate compound, wherein the compositions are in the form of food. As used herein, a “food” is a nutritious solid, semi-solid, liquid, food ingredient or food additive. “Semi-liquid” refers in the context of food to an otherwise solid component dispersed in a liquid milieu, e.g. without limitation, cereal in milk. A food bar or candy bar comprising a pyruvate compound as defined herein (e.g. sodium pyruvate) is an exemplary solid food composition of the invention.

In certain embodiments, the compositions provided by the invention are foods or dietary supplements in the form of a beverage. In certain embodiments, the compositions are foods. In certain embodiments, the compositions are dietary supplements.

In certain embodiments, the present invention provides a food additive or food ingredient comprising a pyruvate compound. “Food additive” or “food ingredient” refers to substances which are not typically ingested per sc, but which arc used in the preparation of food and/or beverages to achieve the benefits provided by the compositions of the invention. Examples of food additive or ingredient include, without limitation, a concentrated form of a composition according to the present invention for mixing with a beverage or food component during preparation thereof.

Examples have been included to more clearly describe particular embodiments of the invention. However, there are a wide variety of other embodiments within the scope of the present invention, which should not be limited to the particular example provided herein.

### EXAMPLES

### Example 1: Striatal Functional Connectivity Predicts Response to Antipsychotic Medications: Findings from Two Independent Cohorts

Currently there is no treatment for Charcot-Marie-Tooth (CMT) neuropathies. Most CMT neuropathy patients, including those with a demyelinating disease of the nerves by histological and electrophysiological criteria at the same time have clinical phenotype of a length-dependent axonal disease. Disruption of energy-dependent axonal transport system as the mechanism underlying distal axonal degeneration has been proposed previously. Fast axonal transport is closely dependent on oxidative phosphorylation and the efficacy of axonal glycolysis is limited in a length dependent fashion. Previous studies showed that pyruvate supplementation allowed the nerve to bypass a blockade in energy production and restored transport by providing an alternative substrate of oxidative metabolism. This example was carried out based on the premise that exogenous pyruvate could have therapeutic value in both demyelinating and axonal CMT neuropathics. The goal is to assess the therapeutic efficacy of sodium pyruvate using functional and electrophysiological studies with quantitative histology in demyelinating (Tr^{J}) mouse models of CMT. Results from these studies will provide evidence that compound muscle action potential (CMAP) can be used as surrogate for clinical/functional improvement as a reliable outcome measure, which has direct relevance to future clinical studies with pyruvate treatment.

### Methods

Six weeks old Tr^{J} mice (pmp22^{Tr-J}) were used in this study. A total of 8 Tr^{J} mice (mouse model for demyelinating CMT) were studied. Five received SP, 4% in drinking water, 3 mice left untreated to serve as control. All treatment protocols and animal surgeries were conducted under the protocols approved by the Nationwide Children's Hospital and The Ohio State University Animal Care and Use Committee. Animals were acclimatized by placing on 2.5% apple juice first, which was increased to 20% a week later. In one group (pyruvate group; n=5) sodium pyruvate (Sigma) was added to apple juice in 2.5% concentration and a week later increased to 4%. The pyruvate group remained 4% sodium pyruvate in 20% apple juice drinking water throughout the duration of the study. The control group (n=3) continued on 20% apple juice only. Drinking solutions were freshly prepared every other day.

Additional studies were designed to assess the efficacy of pyruvate treatment using a quantitative test that has functional significance. Six Tr^{J} mice received SP in their drinking water and the sciatic nerve conduction studies were carried out at baseline and 16 weeks of SP supplementation. Among the parameters obtained from these studies CMAP is a direct measure of the size of the motor unit. Results were compared to age-matched Tr^{J}-control cohort. Functional studies (hind limb grip strength, wire hanging test and rotorod) were done at baseline and at 7 to 10 day intervals.

Surgical Procedures, Tissue Allocation for Morphological Studies.

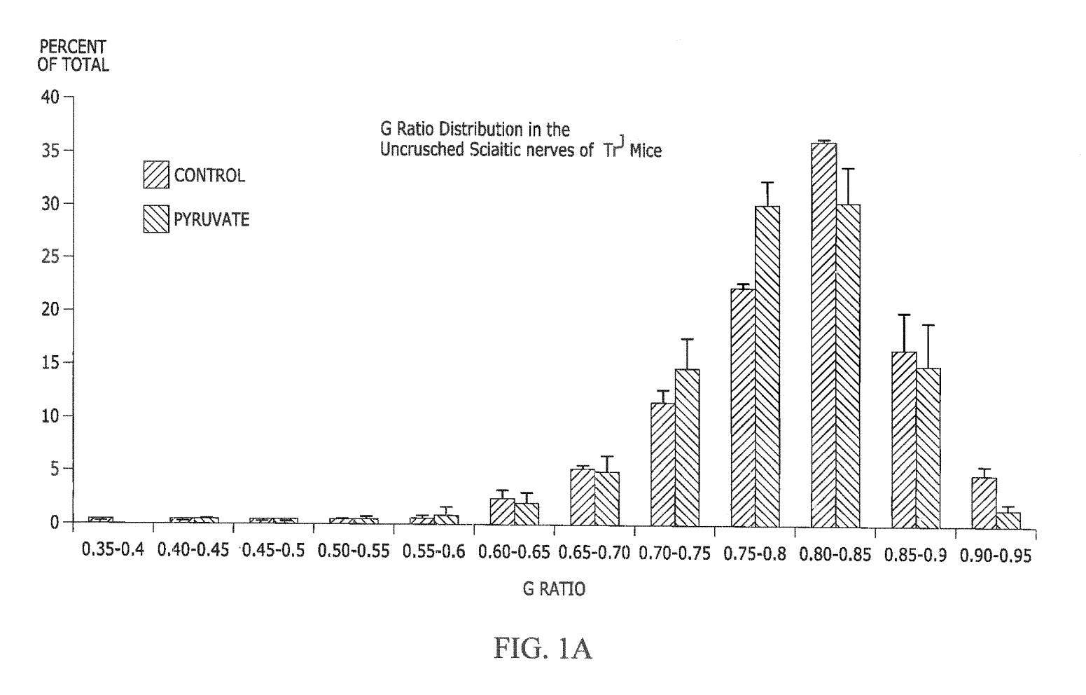

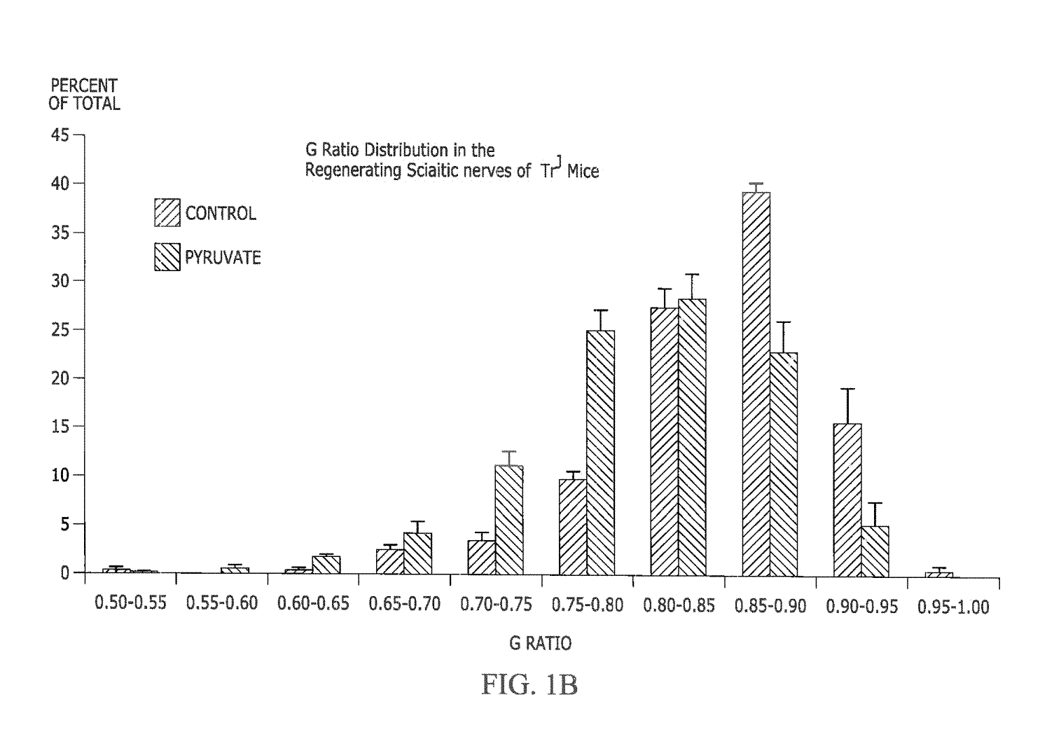

Both groups underwent sciatic nerve crush procedure one week after starting on sodium pyruvate in apple juice or apple juice alone, providing the results shown in FIGS. 1 and 2. Under isoflurane anesthesia left sciatic nerves were exposed and crushed with a fine forceps at a level 5 mm distal to the sciatic notch to generate a regeneration paradigm as previously described. Sahenk et al., Exp Neurol 224, 495-506 (2010). The crush site was marked by a 10-0 nylon suture tie passed through the epineurium. Mice were then killed quickly by an over-dosage of xylazine/ketamine anesthesia and the sciatic nerves from crushed and intact sites were removed under a dissecting microscope. Approximately 2 mm in length tissue blocks immediately distal to the crush site and the subsequent three segments, all marked for proximo-distal orientation as well as the mid sciatic segments from the contralateral uncrushed nerves were processed for plastic embedding for light microscope thick sections and electron microscopy using standard methods established by the inventors.

Myelinated Fiber Density Determinations:

Quantitative analysis at the light microscopic level was performed on 1 μm-thick toluidine blue stained cross sections from regenerating and intact uncrushed sciatic nerves from pyruvate and control groups. Three randomly selected areas of mid-sciatic segments from the intact nerves from the right and the regenerating segments from the left, approximately 3 mm distal to the crush site were photographed using 100× objective and axon diameter measurements were obtained from the computer screen image frames, magnified to 10,384 μm². Composites of fiber size distribution histograms and mean myelinated fiber (MF) densities (mean±SEM) were generated by combining data from all mice. Unpaired t-test was calculated using graphPad Prism 6 program for comparison of the two sets of data.

g Ratio of the Myelinated Fibers:

The g ratio refers to the ratio of axonal diameter/fiber diameter and lower g ratios represent axons with thicker myelin. Beuche, W., and Friede, R. L., Acta Neuropathol 66, 29-36 (1985); Friede, R. L., and Beuche, W., J Neurol Neurosurg Psychiatry 48, 749-756 (1985). For each animal, measurements from all fibers in 3 randomly selected representative unit areas were included and the g ratio distribution histograms were generated as percent of total fibers analyzed as previously described. Measurements from regenerating and intact nerves were obtained in each group. One way ANOVA was used to determine statistical analysis.

### Results

Twenty weeks after crush, in the pyruvate group, microscopic examination of cross sections of sciatic nerve segments showed an increase in the number of MFs in the regenerating and intact sciatic nerves compared to the controls. Quantitative studies confirmed these observations and revealed statistically significant increases in MF densities in the pyruvate (Table 1). FIG. 2 (A, B) shows the composite histograms generated from pyruvate and control Tr^{J} mice. Both in regenerating and intact nerves, the increase in MF densities arc most prominent for those fibers with axonal diameter less than 4 μm. In the regenerating nerves this increase is associated with a shift to larger diameter axons (FIG. 3A). Moreover, G ratio (axon diameter/fiber diameter) determinations of the MFs in the regenerating and intact sciatic nerves showed an increase in myelin thickness indicating that pyruvate supplementation is partially improving the hypomyelination/amyelination state, the hallmark of trembler pathology. The mean G ratio from sciatic nerve in the control Tr^{J} is 0.77±0.003, which is significantly greater than that obtained from wild type (0.66±0.002, p<0.0001), reflecting the hypomyelination state in this model. G ratios were significantly reduced in the pyruvate group, showing a shift of G-ratio to the left, indicative of an increased myelin thickness in comparison to the control Tr^{J} as seen in FIG. 1. The percent of fibers within a G ratio range of 0.4-0.7 constituted about 29% of total fibers in the intact nerves and 22% in the regenerating nerves but was only 6.5% in the control Tr^{J} nerves.

TABLE 1
Myelinated Fiber Density (number/mm2) in the Sciatic
Nerves from Tr^{J} Mice

| Treatment | No. of | Intact | Regenerating |
|---|---|---|---|
| groups | animals | mean ± SEM | mean ± SEM |
| Control | 3 | 13600 ± 325* | 13728 ± 154** |
| pyruvate | 5 | 16583 ± 749* | 18259 ± 1051** |

*p = 0.0271;
**p = 0.0075

The inventors found that SP, 4% in drinking water in Tr^{J} at 20 weeks significantly improved regeneration-associated myelination (g ratio) and myelinated fiber densities in crushed-regenerating as well as uncrushed-intact sciatic nerves. G ratio (axon diameter/fiber diameter) determinations of the myelinated fibers in the intact (FIG. 1A) and regenerating (FIG. 1B) sciatic nerves showed an increase in myelin thickness indicating that pyruvate supplementation is partially improving the hypomyelination/amyelination state, the hallmark of trembler pathology. The mean G ratio from sciatic nerve in the control Tr^{J} is 0.77±0.003, significantly greater than that obtained from wild type (0.66±0.002, p<0.0001), reflecting the hypomyelination state in this model. G ratios were significantly reduced in both intact and regenerating nerves from the pyruvate group, showing a shift of G-ratio to the left, indicative of an increased myelin thickness in comparison to the control Tr^{J} nerves. The percent of fibers with G ratio less than 0.8 constituted about 53% of total fibers in the intact nerves and 43% in the regenerating nerves in the pyruvate group compared to the control trembler nerves, 42% and 17% respectively. These results provide strong morphological evidence for the efficacy of pyruvate supplementation for improving myelination, not only during regeneration (by about 27% more than that of the control group) but also in the intact/uncrushed nerves leading to significant increases in the myelinated fiber densities.

CMAP amplitudes at baseline (BL) and endpoint (EP) in pyruvate (Pyr) and control (Contr) groups are shown in FIG. 3. Pyruvate supplementation for 24 weeks protected CMAP amplitude of the Tr^{J} sciatic nerves; in un-treated Tr^{J} controls significant further deterioration of CMAPs occurred within the same period. Trends for improvements in conduction velocities and distal latencies were not significant.

In addition, motor functions, hind limb grip strength and wire hanging test (WHT) were significantly better in the pyruvate group at 16 weeks of treatment compared to the untreated Tr^{J} controls.

### Example 2: AAV1.NT-3 and Pyruvate Combinatorial Therapy

Charcot-Marie-Tooth (CMT) neuropathies represent a heterogeneous group of peripheral nerve disorders affecting 1 in 2,500 persons. One variant, CMT1A, is a primary Schwann cell (SC) disorder, and represents the single most common variant. In previous studies, the inventors showed that neurotrophin-3 (NT-3) improved the trembler^{J} (Tr^{J}) mouse and also showed efficacy in CMT1A patients. Long-term treatment with NT-3 was not possible related to its short half-life and lack of availability. This led to considerations of NT-3 gene therapy via adeno-associated virus (AAV) delivery to muscle, acting as secretory organ for widespread distribution of this neurotrophic agent. In the TrJ model of demyelinating CMT, rAAV1.NT-3 therapy resulted in measurable NT-3 secretion levels in blood sufficient to provide improvement in motor function, histopathology, and electrophysiology of peripheral nerves. Furthermore, it was shown that the compound muscle action potential amplitude can be used as surrogate for functional improvement and established the therapeutic dose and a preferential muscle-specific promoter to achieve sustained NT-3 levels. These studies of intramuscular (i.m.) delivery of rAAV1.NT-3 serve as a template for future CMT1A clinical trials with a potential to extend treatment to other nerve diseases with impaired nerve regeneration. For further details, see Sahenk et al., Mol Ther. 22(3):511-21 (2014), the disclosure of which is incorporated herein by reference.

AAV Vector Construction.

Vector DNA plasmid pAAV.CMV.NT-3 was used to generate single-stranded rAAV1.CMV.NT-3. It contains the human NT-3 CDS (GeneBank designation NTF3) under the control of the CMV promoter cloned between AAV2 inverted terminal repeats. To generate self-complementary (so) AAV vectors, AAV DNA plasmid vectors pscAAV.CMV.NT-3 were generated as follows: the NT-3 coding sequence was polymerase chain reaction (PCR) amplified from plasmid, the pAAV.CMV.NT-3 vector using forward and reverse primers. The NT-3 PCR fragment was then digested with Not I and ligated into the self-complementary pAAV.CMV.X5 (b54) vector from which the X5 cDNA was removed by Not T digestion. For generating self-complementary DNA vector plasmid pscAAV.tMCKLNT3, the NT-3 cDNA was amplified from plasmid pAAV.CMV.NT-3 by PCR using forward and reverse primers. The resulting NT-3 cDNA PCR fragment was then digested with Kpn I and Asc I enzymes and cloned into a self-complementary pscAAV.tMCK.aSG vector plasmid from which the aSG transgene was removed by Kpn I and Asc I digestion. The final constructs were confirmed by restriction digestion and sequencing. All vectors include a consensus Kozak sequence, an SV40 intron, and synthetic polyadenylation site (53 bp). The tMCK promoter (713 bp) was a kind gift from Dr. Xiao Xiao (University of North Carolina, Chapel Hill, N.C.). Wang et al., Gene Ther., 15:1489-1499 (2008). It is a modification of the previously described CK6 promoter (Shield et al., Mol Cell Biol. 16:5058-5068 (1996)) and includes a modification in the enhancer upstream of the promoter region containing transcription factor binding sites. The enhancer is composed of 2 E-boxes (right and left). The tMCK promoter modification includes a mutation converting the left E-box to a right E-box (2R modification) and a 6 bp insertion (S5 modification).

rAA V Vector Production.

AAV1 vector production was accomplished using a standard 3 plasmid DNA/CaPO₄ precipitation method using HEK293 cells. Two hundred and ninety-three cells were maintained in DMEM supplemented with 10% fetal bovine serum and penicillin and streptomycin. The production plasmids were: (i) pAAV.CMV.NT-3, pscAAV.CMV.NT-3, or pscAAV.tMCK.NT-3 (ii) rep2-cap1 modified AAV helper plasmid encoding the cap 1 serotype, and (iii) an adenovirus type 5 helper plasmid (pAdhelper) expressing adenovirus E2A, E4 ORF6, and VA III RNA genes. A quantitative PCR-based titration method was used to determine an encapsidated vg titer utilizing a Prism 7500 Taqman detector system (PE Applied Biosystems, Grand Island, N.Y.). Clark et al., Hum Gene Ther. 10:1031-1039 (1999). The primer and fluorescent probe targeted the tMCK and CMV promoters.

Animals, Procedures and Treatment Groups.

Tr^{J} mice (B6.D2-Pmp22^{Tr-J}/J) and C57BL/6 wild type were obtained from Jackson Laboratory (Bar Harbor, Me.). All animal experiments were performed according to the guidelines approved by The Research Institute at Nationwide Children's Hospital Animal Care and Use Committee. The design of the experimental groups comparing single-stranded and self-complementary AAV1.NT-3 vectors, treatment duration, doses, and promoters is outlined below: (i) for the nerve regeneration study, 9-12-week-old Tr^{J} mice were injected in the left gastrocnemius muscle with either PBS or 1×10¹¹ vg of asAAV1.CMV.NT-3 (n=12). At 3 weeks postinjection, under isoflurane anesthesia, left sciatic nerves were exposed and crushed with a fine forceps at a level 5 mm distal to the sciatic notch to generate a regeneration paradigm as previously described. Sahenk et al., Ann Neurol., 45:16-24 (1999). Functional recovery, measured weekly by grip strength obtained from the limb harboring the crushed nerve and the morphological assessment of nerve regeneration were the primary endpoints of this study. At 20 weeks, postcrush mice were euthanized for tissue and serum collection for NT-3 ELISA enumeration. (ii) In this set of experiments, the effect of NT-3 gene therapy on the sciatic nerve motor conduction parameters and on the motor functions (ipsilateral and simultaneous bilateral grip strength) were investigated with endpoint correlative histopathology. Six- to 8-week-old Tr^{J} mice received 1×10¹¹ vg of ssAAV1.CMV.NT-3 or PBS in the right quadriceps muscle (n=14 in each group). The left sciatic nerve conduction studies were performed at baseline age and were repeated at 20 and 40 weeks post-gene transfer. At 20 weeks, four vector-injected and five PBS-injected mice were euthanized for tissue collection for the assessment of NF cytoskeleton and NF phosphorylation studies using ultrastructural morphometry and western blot. Functional status of the remainder mice were monitored using rotarod between 23 and 40 weeks, and following endpoint electrophysiology, mice were euthanized for harvesting left sciatic nerve and distal leg muscles. (iii) The efficacy of scAAV1.NT-3 under control of the CMV promoter versus the muscle-specific tMCK promoter both given at three doses, within a half-log range (3×10⁹ vg, 1×10¹⁰ vg, and 3×10¹⁰ vg) was assessed using endpoint electrophysiological and morphological studies. A total of 177 Tr^{J} mice in 7 cohorts (n=23-29 in each cohort) were generated, receiving i.m. injections of the self-complimentary vectors into the right gastric muscle at low dose, intermediate dose, or high dose with either promoters as indicated above or PBS. Technically acceptable quality nerve conduction studies were obtained from the left sciatic nerves in 171 mice. At the end of each study, mice were euthanized for tissue and serum collection for NT-3 ELISA. MF density determinations were done in high-dose cohorts (n=13 with CMV, n=26 with tMCK, and n=12 with PBS).

Using the methods described below, nerve conduction studies were performed at the baseline and the endpoint, 4 months post-AAV1.NT-3 injection. During this period TrJ mice were given ad libitum access to drinking water containing 4% SP. Endpoint CMAP amplitudes in the combinatorial therapy group was significantly better than the group receive SP alone (FIG. 4).

Functional studies at the end point are shown in FIG. 5, which provides bar graphs comparing the results of combination therapy with those obtained using pyruvate alone. Both pyruvate (n=6) and pyruvate+NT-3 (n=10) treated group performed better than control group (n=6) in both test (*P<0.05).

Combination therapy improved trembler pathology significantly compared to untreated controls. FIG. 6 provides images of m thick toluidine blue stained plastic sections from sciatic nerves of TrJ, untreated (UnTr), treated with sodium pyruvate (SP) alone (PYR) and received the combination of AAV1.NT-3 gene therapy and SP (PYR+NT3) at 16 weeks of treatment. Untreated TrJ mice nerves show severe hypomyelination. A notable increase in myelinated fibers and increased myelin thickness is seen in both treatment groups, which was more prominent in the nerves received the combination therapy.

Morphometric studies corroborated the functional and electrophysiological studies showing significant increases in MF densities in the sciatic nerves from both treatment groups compared to the untreated TrJ-controls. The results are shown in Table 2.

TABLE 2
Myelinated Fiber Density (number/mm2) in
the Sciatic Nerves from Tr^{J} Mice

| Treatment | Treatment | No. of | |
|---|---|---|---|
| groups | Duration | animals | mean ± SEM |
| Control | 16 wks | 6 | 12377.9 ± 918.3 |
| PYR | 16 wks | 6 | 15000.2 ± 878.4* |
| PYR + NT3 | 16 wks | 6 | 15782.03 ± 1099.4** |

*p < 0.0003, vs. control;
*p < 0.0001, vs. control.

Axon diameter distribution histograms of myelinated fibers showed a more prominent shift to larger diameter axons in the combinatorial therapy cohort indicating that pyruvate and NT-3 has synergistic effect in radial growth of axons compared to the cohort treated with pyruvate only.

### Methods

Wire Hang test: The mice were allowed to grasp by their four paws a 2-mm diameter metal wire maintained horizontally 35 cm above a thick layer of soft bedding (device made by the NCH Bioengineering Department according to the specifications of Klein et al.). Klein et al. J Neurosci Methods, 203:292-297 (2012). The length of time until the mice fell from the wire was recorded. After each fall, the mice were allowed to recover for 1 minute. Each session consisted of three trials from which the scores were averaged.

Motor function testing Tr^{J} mice were tested for baseline motor function within 1 week prior to receiving i.m. injection of ssAAV1.CMV.NT-3 or PBS. Motor function tests included bilateral simultaneous hindlimb grip power and that of the left hind paw using a grip strength meter (Chatillon Digital Meter; Model DFIS-2; Columbus Instruments, Columbus, Ohio). Bilateral or unilateral grip strength was assessed by allowing the animals to grasp a platform followed by pulling the animal until it releases the platform; the force measurements were recorded in four separate trials. Measurements were performed on the same day and time of each week. Endpoint bilateral and ipsilateral grip strength measurements were done in two sessions (morning and afternoon), three trials in each per day for 3 consecutive days prior to obtaining the nerve conduction studies. The mean of these measurements were used to correlate with conduction studies.

Nerve Conduction Studies: Sciatic motor nerve conduction studies were performed on mice under isoflurane anesthesia using a portable electrodiagnostic system (Synergy N2 EMG and nerve conduction study machine, Natus, Middleton, Wis.) as previously described. Yalvac et al., Mol Ther. 22(7):1353-63 (2014). Distance for electrode placement was measured using a compass with a needle edge and a millimeter-graduated tape measure. The sciatic motor nerve conduction responses were recorded using two fine ring electrodes (Alpine Biomed, Skovlunde, Denmark) used as the active (E1) and reference (E2) electrodes. The active recording electrode was placed over the mid portion of the gastrocnemius muscle and the reference electrode over the tendon. A pair of 28 gauge monopolar needle electromyography electrodes (Teca, Oxford Instruments Medical, New York, N.Y.) was used to provide supramaximal stimulus to the sciatic nerve at the distal thigh and sciatic notch. The parameters measured included compound muscle action potential (CMAP) amplitude, distal latency, and conduction velocity.

### Histopathological Studies:

Myelinated fiber density determinations. Quantitative analysis at the light microscopic level was performed on 1 μm-thick cross sections from regenerating and intact uncrushed sciatic nerves using images captured at ×100 magnification and evaluated by image analysis software (Bioquant TCW98 and 2014 updated version of image analysis software, R&M Biometrics Inc., Nashville, Tenn.) as previously described. Sahenk et al., J Peripher Nerv Syst 8: 116-127 (2003). Data assessing regeneration response were obtained from the second segment, at a level approximately 4 mm distal to the crush. The mid sciatic nerve segments were analyzed from uncrushed intact nerves in all cases. Four randomly selected areas were analyzed in each mouse. MF densities (mean number+SE/mm²) and composites of myelinated fiber axon size distribution histograms were generated in treated and untreated cohorts for comparison. A composite histogram showing myelinated fiber distribution in the treated and untreated sciatic nerves from TrJ mice at 16 weeks of treatment is shown in FIG. 7.

g ratio of the myelinated fibers: The g ratio refers to the ratio of axonal diameter/fiber diameter and lower g ratios represent axons with thicker myelin. For g ratio determinations, 2 to 3 representative areas of cross sectional images of mid sciatic nerves from 3 TrJ mice in each cohort were captured at ×100 magnification, and the shortest axial lengths as axon diameters and fiber diameters were recorded with a calibrated micrometer, using the AxioVision, 4.2 software (Zeiss) as we described previously. Sahenk et al. Experimental neurology 224: 495-506 (2010). The g ratio distribution histograms were generated as percent of total fibers analyzed.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method of treating a subject having peripheral neuropathy, comprising administering a therapeutically effective amount of a pyruvate compound to the subject, wherein the subject has been diagnosed as having a disorder of the peripheral nervous system exhibiting muscle weakness, atrophy and/or sensory dysfunction.

2. The method of claim 1, wherein the pyruvate compound is a pharmaceutically acceptable pyruvate salt.

3. The method of claim 2, wherein the pyruvate compound is selected from the group consisting of sodium pyruvate, calcium pyruvate, potassium pyruvate, and magnesium pyruvate.

4. The method of claim 2, wherein the pyruvate compound is sodium pyruvate.

5. The method of claim 1, wherein the pyruvate compound is a pyruvate alkyl ester derivative.

6. The method of claim 1, wherein the pyruvate compound is administered over a substantial period of time.

7. The method of claim 1, wherein the pyruvate compound is administered in a plurality of separate administrations.

8. (canceled)

9. The method of claim 1, wherein the peripheral neuropathy is Charcot-Marie-Tooth neuropathy.

10. The method of claim 1, wherein the pyruvate compound is administered in a controlled release formulation.

11. The method of claim 1, wherein the pyruvate compound is orally administered.

12. The method of claim 1, wherein the pyruvate compound is a pharmaceutically acceptable salt that is orally administered in a controlled release formulation.

13. The method of claim 1, wherein the method of treatment consists of administering a therapeutically effective amount of a pyruvate compound in a pharmaceutically acceptable formulation to the subject over a substantial period of time.

14. The method of claim 1, further comprising gene therapy treatment of the peripheral neuropathy.

15. The method of claim 14, wherein the gene therapy treatment increases neurotrophin-3 expression.

16. The method of claim 14, wherein the gene therapy is carried out using a recombinant adeno-associated virus mediated gene transfer.

17. A controlled release formulation of a pharmaceutically acceptable pyruvate salt.
